(19) United States
(12) Patent Application Publication
(10) Pub. No.: US 2004/0265785 A1
(43) Pub. Date: Dec. 30, 2004

(54) Method for treating materials of biological origin, and collagen-elastin product

(75) Inventors: Leon Olde Damink, Bunde (NL); Ingo Heschel, Herzogenrath (DE)

Correspondence Address: HENRY M FEIEREISEN, LLC, 350 FIFTH AVENUE, SUITE 4714, NEW YORK, NY, 10118, US

(73) Assignee: MATRICEL GMBH, Herzogenrath (DE)

(21) Appl. No.: 10/853,766

(22) Filed: May 24, 2004

Related U.S. Application Data
(63) Continuation-in-part of application No. PCT/DE02/04278, filed on Nov. 21, 2002.

(30) Foreign Application Priority Data
Nov. 22, 2001 (DE) 101 57 182.8

(51) Int. Cl. A01N 1/02; C12N 1/08

(52) U.S. Cl. 435/1.1; 435/270

(57) ABSTRACT

In order to achieve a reproducible treatment for materials of biological origin such as membranes, skins, vessels, heart valves, tendons and ligaments and, in particular, collagen materials, hydrophobic accompanying substances are, first of all, chemically removed without the accompanying substances being chemically altered. Non-hydrophobic accompanying substances are subsequently removed. The materials treated in this manner are used to form membranes, heart valves or vessels or are further processed to form solutions, fibers, fiber weaves, fibrous tissues or sponges and are optionally populated with cells and/or used for carrying out controlled tissue regeneration.

### CROSS-REFERENCES TO RELATED APPLICATIONS

This application is a continuation-in-part of prior filed copending PCT International application no. PCT/DE02/04278, filed Nov. 21, 2002, which designated the United States and on which priority is claimed under 35 U.S.C. §120, the disclosure of which is hereby incorporated by reference, and which PCT International application no. PCT/DE02/04278, filed Nov. 21, 2002 claims the priority of German Patent Application, Serial No. 101 57 182.8, filed Nov. 22, 2001, pursuant to 35 U.S.C. 119(a)-(d), the disclosure of which is incorporated herein by reference.

### BACKGROUND OF THE INVENTION

The present invention relates to a method for treating materials of biological origin such as membranes, skins, vessels, heart valves, tendons and ligaments, from which hydrophobic accompanying substances are removed. More particularly, the present relates to a purification method for collagen materials which can be used without changing the original collagen architecture, or can be reshaped into other structures. The present invention also relates to an elastin product.

To ensure clarity, the term “collagen materials” as used throughout this disclosure relates to materials of biological origin some of which are composed of more than one structural protein and supportive proteins. It is possible that not only collagen but also elastin is present in considerable amounts which can even be favorable for certain applications. Typically, these collagen materials are harvested from animal membranes like intestine, Fascia Lata, pericardium, peritoneum, omentum or dura mater. Other examples are the purification of heart valves or blood vessels. In addition, skins, ligaments like ligamentum nuchea, ligamentum cruciatum and tendons like Achilles tendon can be processed into collagen fibers. These fibers can either be applied directly or be formed into wound covers, sponges, cell carrier systems and the like.

A method for the preparation of collagen materials is described in the PhD thesis of A. Ghofrani, Development of an autologous split skin equivalent, Dissertation RWTH Aachen, 27 Apr. 1998, Shaker-Verlag, Aachen. Materials manufactured according to the described process had unacceptable variability in cell culture and biocompatibility experiments and some times were poorly resistant to enzymatic breakdown making the materials not practically applicable. This observation could be correlated to the fact that the human and animal source materials used as a starting material not only contain collagen but also other substances like cells and non-collagenous substances. Before the collagen structures can be applied in or on a patients body, these cells and non-collagenous substances need to be removed to guarantee a harmless tissue reaction and prevent immune reactions from occurring. Examples of non-collagenous substances are non-structural proteins, proteoglycans composed of proteins and glycosaminoglycans as well as lipids. The lipids can be divided into fatty acid type lipids or steroid type lipids. Removal of these substances needs to be performed in a delicate fashion, which does not affect the helical structure of the collagen.

According to U.S. Pat. No. 5,028,695, the first step in the purification process of the starting materials is the mechanical removal of the lipids followed by the treatment with a strong alkaline solution until the amido nitrogen content is below 0.35 mmol/g or less. Next, the materials are treated with strong acids and if needed enzymes, removing accompanying contaminations from the collagen raw material.

International patent publication no.: WO 90/03811 describes a process in which pericardial membranes are mechanically defatted followed by a treatment with an alkaline solution, a sodium chloride solution, a complexing agent and an acid buffer. In the last step of the process, the membranes are defatted with acetone.

According to International patent publication no: WO 95/18638, membranes are purified using an alkaline soak, an acidic soak and a water wash and finally dried. In the final process step, they are defatted.

All processes described above are based on the removal or solubilization of cell residues and non-collagenous substances in an aqueous environment. This is applicable to the processes involving a chemical action as well as an enzymatic action for the removal of cell and accompanying substances. The processes described are based on three process steps: first the starting materials are mechanically or during alkaline hydrolysis defatted. Thereafter, cells and other accompanying substances are removed and finally, the materials are dried and in some cases defatted after drying. The second step in the process, the removal of cells and other accompanying substances is performed in an aqueous environment and for this process step it is very important that the aqueous solutions not only penetrate the collagen structure on a macroscopical scale but also on a microscopical and molecular scale to guarantee a complete and in-depth purification of the collagen structures. This is not achieved using the methods described above.

The presence of significant amounts of lipids in the deeper layers of the collagen structure forms the basis of the present invention. These lipids are hydrophobic and will prevent the penetration of the aqueous solution on a molecular scale. It is described in some of the publications identified above that part of the lipids are mechanically removed before the aqueous treatments are started. This can however not guarantee that the mechanical defatting is sufficient to achieve a complete purification. On the contrary, mechanical defatting could result in the distribution of the lipids into the deeper layers of the collagen structures due to the mechanical action. In addition, mechanical defatting could not be applicable to delicate collagen structures since it could damage these delicate collagen structures.

It was suggested to use alkaline conditions during the purification to hydrolyze the lipids followed by removal through washing. This can only be used in case of fatty acid type lipids but not in case of steroid type lipids. In addition, the hydrolysis of fatty acid type lipids under the conditions typically used during the alkaline purification of collagen structures is very slow and will never result in complete removal of these lipids.

As a consequence, when using conventional processes, the purity of the collagen structures depends on associated phenomena that are difficult to control and result in a poor reproducibility of the purely mechanical and alkaline treatment. Thus the collagen structures prepared in this manner are not optimally suited for use as cell scaffolds and as a matrix for tissue reconstruction/regeneration. More particularly, the cell cultivation is not sufficiently reproducible.

It would therefore be desirable and advantageous to provide an improved method for treating materials of biological origin, which obviates prior art shortcomings and is able to specifically purify collagen materials without changing the original collagen architecture.

### SUMMARY OF THE INVENTION

The procedure described in this invention involves an effective method for the removal of lipids and hydrophobic substances from tissue structures. According to one aspect of the invention, a process for treatment of materials of biological origin, such as membranes, skins, blood vessels, heart valves, tendons and ligaments and, in particular, collagen materials, includes the steps of chemically removing from the materials hydrophobic accompanying substances without the accompanying substances being chemically altered, and subsequently removing non-hydrophobic substances.

By this method, the lipids are completely removed in the first step, without the need for chemical modification of the lipids. Thereafter, an aqueous purification can be performed without the potential issue that parts of the collagen structures are not accessible for aqueous solutions due to the presence of hydrophobic substances.

It is favorable that the collagen materials are disinfected before the removal of the hydrophobic substances.

In one of the process variants, the hydrophobic substances are removed through a wash with a water miscible organic solvent like for instance alcohols or acetone. This allows the removal of the lipids without the need for initial chemical modification of the lipids. Also mixtures of water miscible solvents with each other or with water lead to positive processing results.

It is favorable that the treatment with water miscible organic solvents is followed with a treatment with water immiscible organic solvents like hexane, diethyl ether, chloroform or petroleum benzene.

Another possibility for the removal of hydrophobic substances is a soak using surfactants. In this case preferable biological acceptable anionic, cationic or non-ionic surfactants are used. Examples of such surfactants are triton X-100, tripolyphosphate, or surfactants from the Tween family, for instance Tween 20 (polyoxyethylene sorbitan monooleate).

Also combinations of water miscible organic solvents (e.g. acetone) and surfactants are possible. Favorable is a solution of 1.0% Tween 20 in a 50% (v/v) acetone/water mixture.

Non-hydrophobic substances can be removed using a solution capable of breaking hydrogen bonds, especially a solution of urea.

In addition, non-hydrophobic substances can be removed using aqueous solutions of inorganic salts, especially buffered sodium potassium or calcium chloride solutions containing enzyme inhibitors.

In a further embodiment, it is described that non-hydrophobic substances can be removed using alkaline solutions for instance sodium, potassium or calcium hydroxide solutions.

In addition, removal of non-hydrophobic substances can be achieved using acidic solutions like hydrochloric acid, sulfuric acid or acetic acid.

Especially good results were obtained through a combination of the following steps: Treatment with a solution removing hydrogen bonded substances followed by a treatment with a solution removing alkali sensitive substances and finally a treatment with a solution removing acid sensitive substances. This combined treatment protocol will have a comparable efficiency in removing non-hydrophobic substances when the sequence of the steps is changed. In addition, in specific cases, some of the steps might be deleted from the process.

It is favorable when the materials of biological origin are confectioned to purified membranes, heart valves or blood vessels or into collagen solutions, collagen fibers, collagen weaves or collagen sponges. Confectioning includes activities to trim, widen or chemically crosslink the treated materials.

A nativity of the biological materials below 95% has proven to be beneficial for many applications. In case of cartilage regeneration, better results were obtained with membranes having a nativity around 80% when compared to membranes having a nativity above 95%. The process according to this invention allows, based on the high reproducibility, to use the resulting materials also for cell culturing matrices. Collagen materials treated according to the process of the present invention have been seeded with skin cells, nerve cells and especially cartilage cells.

The processed materials of biological origin and the confectioned materials can be used for tissue engineering purposes. Especially in case of joint cartilage repair using the ACI or MACI procedures (Autologous Chondrocyte Implantation respectively Matrix-induced Autologous Chondrocyte Implantation), the suitability of collagen materials produced according to the present invention have been shown in in-vitro cell culture experiments and animal implantation studies.

The problems associated with materials produced according to the current state of the art are also solved with a collagen-elastin product composed of insoluble collagen and insoluble elastin. By adding insoluble elastin to for instance sponges results in a slower resorption and therefore in an increased resistance of the processed material. In addition, the calcification of such collagen-elastin products in-vivo is significantly reduced. Finally, the addition of insoluble elastin results in an increased elasticity and improved strength. In case the elastin is already present in the material of biological origin, the advantage of the insoluble elastin is inherent in case of processing the materials according to the current invention.

The realization of the advantages of the present invention can best be attained when insoluble elastin is added. Otherwise it will be resorbed too quickly and a permanent improvement in mechanical properties is not possible.

Especially advantageous results are obtained when of the elastin used, at least 20% of the elastic fibers contain the originally present fibrillin structure. In such collagen-elastin products, the elastic fibers containing the fibrillin structures are microscopically or histologically detectable.

In case of additional processing of the elastin to remove the fibrillin structures, the resulting elastin preparations lose their initially positive properties. As a consequence, the advantage of the present invention is lost more and more in case of additional processing of the elastin and is completely lost in case of the use of soluble elastin.

### DETAILED DESCRIPTION OF PREFERRED EMBODIMENTS

Examples of the method for treating materials of biological origin according to this invention and the special advantages are described below.

### EXAMPLE 1

Maximally 4 kg of pericardial membranes are disinfected and inactivated for minimally 1 hour in 50 liter of a 1.0 N sodium hydroxide solution. The temperature of the alkaline solution is 20° C. At the end of the disinfection period, a hydrogen chloride solution is added to neutralize the alkaline solution (pH between 6.0 and 8.0). The membranes are washed 3 times with water to remove residues of the neutralization process. Thereafter, the membranes are defatted with 3 changes of a 50 liter acetone bath. The acetone is removed by washing the membranes 3 times with water.

The membranes are treated for 1 to 21 days with a 0.05 N-0.1 N solution of sodium hydroxide. The pH of this solution is between 12 and 14 during the alkaline treatment. A hydrogen chloride solution is used for neutralization of the alkaline solution followed by a water wash for 3 times. Finally, the membranes are dehydrated using acetone and dried on air. The nativity of the materials can be set to the desired value using ⁶⁰Co gamma irradiation with a dose between 10 kGy and 40 kGy. The dried product is composed of a fibrous collagen network, having the native fiber arrangement.

After confectioning and sterilization, these membranes can be used as reinforcement or implant material during surgical procedures, as a membrane for tissue engineering purposes or as a carrier for cell culturing. The mechanical properties and the rate of resorption of these membranes can be optimized by chemical crosslinking methods. Applicable methods include the use of glutaraldehyde-, hexamethylene diisocyanate-, carbodiimide- or polyepoxide-based crosslinking methods.

### EXAMPLE 2

Maximally 4 kg of peritoneal membranes are disinfected and inactivated for minimally 1 hour in 50 liters of a 1.0 N sodium hydroxide solution. The temperature of the alkaline solution must be 20° C. Sodium chloride can be added to control the swelling of the membranes.

After disinfection, a hydrogen chloride solution is added to achieve a pH between 6.0 and 8.0. The peritoneal membranes are washed 3 times with water. Thereafter, the membranes are defatted with 3 changes of a 50 liter acetone bath. Effective defatting can be achieved when the acetone is heated to temperatures above 35° C. for a minimum of 30 minutes. The acetone is removed by washing the membranes 3 times with water.

The membranes are treated for 1 to 21 days with a 0.05 N-0.1 N solution of sodium hydroxide. The pH of this solution is between 12 and 14 during the alkaline treatment. A hydrogen chloride solution is used for neutralization of the alkaline solution followed by a water wash for 3 times.

The membranes are treated for 1 to 21 days with a 0.05 N-0.1 N solution of hydrogen chloride. A sodium hydroxide solution is used for neutralization of the acidic solution followed by a water wash for 3 times. Finally, the membranes are dehydrated using 2×50 liters of acetone for 30 to 60 minutes and dried on air.

The nativity of the materials can be set to the desired value using ⁶⁰Co gamma irradiation with a dose between 5 kGy and 50 kGy.

As shown below, membranes are more effectively defatted when the acetone is heated. The preferred heating temperature should be selected to be above the melting point of the lipids to be removed.

| | Lipid Content |
|---|---|
| After manual defatting | Typically 20% |
| | (measured values 20.3% - 19.1%) |
| After acetone defatting, without | Typically 3.0% |
| heating | (measured values 3.51% - 2.28%) |
| After acetone defatting, with heating | Measured values < 1.0% |
| After acetone + hexane, without | Measured values < 0.5% |
| acetone heating | |

For example, manually defatted membranes had a lipid content of 20.3% and 19.1%, which was reduced to <1.0% by treatment in heated acetone.

The dried product is composed of a fibrous collagen-elastin network, in which the native fiber arrangement is retained. The elastin fibers still contain the fibrillin structure that was originally present.

After confectioning and sterilization these membranes can be used as reinforcement or implant material during surgical procedures, as a membrane for tissue engineering purposes or as a carrier for cell culturing. The mechanical properties and the rate of resorption of these membranes can be optimized by chemical crosslinking methods. Applicable methods include the use of glutaraldehyde-, hexamethylene diisocyanate-, carbodiimide- or polyepoxide-based crosslinking methods.

### EXAMPLE 3

Maximally 4 kg of tendons are disinfected and inactivated for 1 hour in 50 liter of a 1.0 N sodium hydroxide solution. The temperature of the alkaline solution must be 20° C. After 1 hour a hydrogen chloride solution is added for neutralization. The tendons are washed 3 times with water until all residues of the neutralization are removed. The tendons are comminuted to optimize the defatting process. The tendons are defatted with 3×50 liter acetone followed by washing with water (3×) to remove all residues of the acetone treatment. During the acetone defatting, heating of the acetone may facilitate the removal of the lipid residues.

The tendons are washed with buffered solutions of sodium chloride containing enzyme inhibitors like ethylene diamine tetra acetic acid (EDTA) or N-ethylmaleimide (NEM). The tendons are washed 3 times with water. The tendons are treated with a 4 M solution of urea followed by 3 times washing with water to remove residues of the urea solution. The tendons are treated with a 0.05 N-0.1 N (0.2%-0.4%) solution of sodium hydroxide for 1 to 21 days. During this time, the pH value of the solution is between 12 and 14.

A hydrogen chloride solution is used for neutralization. The tendons are washed 3 times with water. The tendons are treated for 1 hour to 7 days with a 0.05 N-1.0 N solution of hydrogen chloride. The pH of this acidic solution is between 0 and 3 during this time period. An alkaline solution is added to neutralize the acidic solution followed by 3× washing with water. The tendons are dried using acetone or by lyophilization.

It has been found experimentally that the defatting process of tendons is more effective when the acetone is heated. For example, manually defatted tendons had a lipid content of 3.30% and 3.46%, which was reduced to <0.6% by treatment in heated acetone, before treatment with alkaline and acidic solutions.

Purification of Tendons

| | | According to | |
|---|---|---|---|
| | Fresh tendons | invention | Commercially |
| Marker molecule | (n/1000n) | (n/1000n) | available (n/1000n) |
| Glucosamine | 0.9 | <0.5 | 0.8 |
| Galactosamine | 0.8 | <0.5 | 0.7 |
| Cysteine | 1.3 | 0.2 | 0.8 |
| Tryptophane | 0.4 | 0.1 | 0.3 |

The amount of glucosamine, galactosamine, cysteine and tryptophane is presented as number of molecules per 1000 molecules (n/1000n). The comparison shows that marker molecule residues for the commercially processed tendons are much higher than for the inventive process (e.g. 4-fold cysteine content, 3-fold tryptophane content).

Using this method for the treatment of materials of biological origin results in purified collagen fibers that can be used for the production of fiber weaves, fiber fleeces or sponges or can be used as a coating material for instance for synthetic blood vessel prostheses.

The mechanical properties and the rate of resorption of these products can be optimized by chemical crosslinking methods. Applicable methods include the use of glutaraldehyde-, hexamethylene diisocyanate-, carbodiimide- or polyepoxide-based crosslinking methods.

After confectioning and sterilization these products can be applied in the field of hemostasis, as a dermal repair matrix in the field of chronic and acute wounds and as a general carrier structure for tissue engineering applications.

### EXAMPLE 4

Maximally 4 kg of neck ligaments (for instance ligamentum nuchea) is treated with 50 liters of a 1.0 N sodium hydroxide solution. The temperature of the alkaline solution must be 20° C. After 1 hour a hydrogen chloride solution is added for neutralization. The neck ligaments are washed 3 times with water until all residues of the neutralization are removed. The neck ligaments are comminuted to optimize the defatting process. The neck ligaments are defatted with 3×50 liter acetone followed by washing with water (3 ×) to remove all residues of the acetone treatment.

The neck ligaments are washed with buffered solutions of sodium chloride containing enzyme inhibitors like ethylene diamine tetra acetic acid (EDTA) or N-ethylmaleimide (NEM). The neck ligaments are washed 3 times with water. The neck ligaments are treated with a 4 M solution of urea followed by 3 times washing with water during 30 to 60 minutes to remove residues of the urea solution. The neck ligaments are treated with a 0.05 N-0.1 N (0.2%-0.4%) solution of sodium hydroxide during 1 to 21 days. During this time, the pH value of the solution is between 12 and 14.

A hydrogen chloride solution is used for neutralization (pH value between 6.0 and 8.0). The neck ligaments are washed 3 times with water.

The neck ligaments are purified for 1 hour to 7 days with a 0.05 N-1.0 N solution of hydrogen chloride. The pH of this acidic solution is between 0 and 3 during this time period. An alkaline solution is added to neutralize the acidic solution followed by 3× washing with water.

The neck ligaments are dried using acetone or by lyophilization. This method for the treatment of materials of biological origin provides an optimal mixture of purified elastin fibers and purified collagen fibers. The elastin fibers still contain the fibrillin structures originally present. This mixture can be used for the production of fiber weaves, fiber fleeces or sponges or can be used as a coating material for instance for synthetic blood vessels prosthesis. In addition, this mixture can also be used after addition of collagen fibers as described in example 3 for the production of fiber weaves, fiber fleeces or sponges or can be used as a coating material for instance for synthetic blood vessel prostheses.

The neck ligament fibers can be used for the production of fiber weaves, fiber fleeces or sponges or can be used as a coating material for instance for synthetic blood vessel prostheses. The mechanical properties and the rate of resorption of these products can be optimized by chemical crosslinking methods as described above.

The products made from these materials can be applied especially in those fields were a combination of elastin and collagen is beneficial. This is especially the case in the field of wound healing as a dermal repair matrix and for the stimulation of neo-vascularization and improvement of the elasticity in the general area of tissue engineering. Products based on this method for the treatment of materials of biological origin can be used in various scaffold structures for tissue engineering applications.

The advantages of the materials of biological origin treated according to one of the above examples will be explained through individual examples below.

During the preparation of collagen membranes not using the initial step for the removal of hydrophobic substances according to this invention, it was established that the amount of lipids present was between 15% and 20% even after the treatment step using an acid. When processed according to the present invention with 3 consecutive acetone baths, the amount of lipids present is typically less than 0.5% so that an optimal accessibility of the collagen membrane is guaranteed during the following aqueous purification steps.

In the table below, the typical values found during characterization of collagen membranes prepared according to the present invention are presented.

| Parameter | Typical value |
|---|---|
| Denaturation temperature | 45-47° C. |
| Nativity* | 78-82% |
| Ash residue | 0.1-0.2% (w/w) |
| Moisture content | 10-15% |
| Amido nitrogen content | 0.29-0.32 mmol/g |
| Lipid content | <0.5% (w/w) |
| Elastin content | 5-30% (w/w) |

*The nativity of the membranes was determined according to the method of Bank as described in detail in: Bank R.A. et al: A simplified measurement of degraded collagen in tissues: Application in healthy, fibrillated and osteoarthritic cartilage, Matrix Biology 16, 233-243 (1997). To achieve a nativity as given in the table above, an irradiation dose of 35 kGy was used.

The purity of collagen structures can be related to the presence of residues of glucosamine and galactosamine. These molecules are characteristic markers for the presence of glucosaminoglycans, i.e. non-collagenous molecules, that need to be removed during the purification process. In the table below the glucosamine and galactosamine content of freshly harvested collagen membranes from the slaughterhouse, commercially available membranes made according to the state of the art (no chemical defatting before aqueous purification) and membranes purified according to the present invention is compared. The glucosamine and galactosamine content was determined after 6 M acid hydrolysis of the material using an amino acid analyzer. The amount of glucosamine and galactosamine is presented as number of molecules per 1000 molecules (n/1000 n).

| | | Membrane | Commercially |
|---|---|---|---|
| | Fresh | according to | available |
| Marker molecule | membrane | present invention | membrane |
| glucosamine | 3.6 | <0.5 | 1.1 |
| galactosamine | 0.5 | <0.5 | 0.6 |

The membranes that are processed according to the inventive process contain less than 0.5 n/1000 n which corresponds to the detection level of 0.5 n/1000 n of the performed analyses.

The direct comparison between the commercially available membrane and the membrane produced according to the invention shows that the inventive process leads to glucosamine concentrations that are at least 55% lower than the concentration for the commercially available membranes.

The effectiveness as a scaffold for cultivation of cells of the commercially available membrane and the membrane made according to the present invention were tested using chondrocytes. In the table below, the data obtained (cell number and cell vitality) after a 3 day culturing period are presented. All membranes were seeded with identical cell numbers from the same cell suspension followed by cultivation under identical conditions.

| | Membrane according to | | Commercially available | |
|---|---|---|---|---|
| | present invention | | membrane | |
| | | Cell vitality | | Cell vitality |
| Experiment | Cell number | (%) | Cell number | (%) |
| 1 | 55750 | 97.4 | 43500 | 96.6 |
| 2 | 51500 | 97.1 | 49000 | 98.0 |
| 3 | 75000 | 96.7 | 40500 | 97.5 |
| 4 | 52500 | 98.1 | 43000 | 96.5 |
| 5 | 54500 | 97.2 | — | — |

It is clearly demonstrated that the cell number is higher on the membranes prepared according to the present invention when compared to the commercially available membranes. The cell vitality is comparable for both membrane types.

During the cell cultivation studies it was observed that the suitability of the membranes prepared according to this invention for this application is better in case the nativity of the membranes is lower then is technically achievable. This leads to the not directly obvious conclusion that membranes with a lower nativity have better cell cultivation properties when compared to membranes with a significantly higher nativity. Especially for the articular cartilage repair using autologous chondrocytes based on collagen membranes prepared according to the current invention, it can be beneficial to adjust the nativity to lower values than maximally achievable using this manufacturing process. One method for lowering the nativity of the products is the use of longer treatment times during alkaline or acidic treatment. Another method is the use of different doses of gamma irradiation.

The membranes prepared according to the present invention were used in implantation studies in mice and rats. Histopathological examination of retrieved tissue samples revealed good biocompatibility and vascularization of the implanted materials with no signs of toxicity or allergic reactions after implantation.

While the invention has been illustrated and described in connection with currently preferred embodiments shown and described in detail, it is not intended to be limited to the details shown since various modifications and structural changes may be made without departing in any way from the spirit of the present invention. The embodiments were chosen and described in order to best explain the principles of the invention and practical application to thereby enable a person skilled in the art to best utilize the invention and various embodiments with various modifications as are suited to the particular use contemplated.

What is claimed as new and desired to be protected by Letters Patent is set forth in the appended claims and includes equivalents of the elements recited therein:

What is claimed is:

1. A method for treatment of a material of biological origin, such as membranes, skins, blood vessels, heart valves, tendons and ligaments and, in particular, collagen materials, comprising the steps of chemically removing from the material hydrophobic accompanying substances without chemical alteration to the accompanying substances; and subsequently removing non-hydrophobic accompanying substances.

2. The method of claim 1, wherein the material is of biological origin, and further comprising the step of disinfecting the material before removal of the hydrophobic accompanying substances.

3. The method of claim 1, wherein the hydrophobic accompanying substances are removed using a wash with a water miscible organic solvent.

4. The method of claim 3, wherein the water miscible organic solvent is ethyl alcohol or acetone.

5. The method of claim 4, wherein the water miscible organic solvent is heated to a temperature above the melting point of lipids to be removed.

6. The method of claim 5, wherein the water miscible organic solvent is heated to a temperature above 35° C. for a minimum of 30 minutes.

7. The method of claim 6, wherein the residual concentration of the hydrophobic accompanying substances after initial chemical purification is below 1.0% (w/w).

8. The method of claim 3, wherein the wash step with water miscible organic solvent is followed by a wash with non-water miscible organic solvent.

9. The method of claim 8, wherein the non-water miscible organic solvent is hexane, diethyl ether, chloroform or petroleum benzene.

10. The method of claim 1, wherein hydrophobic accompanying substances are removed using a wash with surfactants.

11. The method of claim 1, wherein the non-hydrophobic accompanying substances are removed using a solution of hydrogen bond breaking substances.

12. The method of claim 11, wherein the solution is a solution of urea.

13. The method of claim 1, wherein the non-hydrophobic accompanying substances are removed using an aqueous solution of inorganic salts.

14. The method of claim 13, wherein the aqueous solution of inorganic salts is selected from the group consisting of a buffered solution of sodium chloride, potassium chloride, and calcium chloride.

15. The method of claim 13, wherein the aqueous solution of inorganic salts, contains an enzyme inhibitor.

16. The method of claim 1, wherein the non-hydrophobic accompanying substances are removed using an alkaline solution.

17. The method of claim 15, wherein the alkaline solution is selected from the group consisting of sodium hydroxide solution, potassium hydroxide solution, and calcium hydroxide.

18. The method of claim 1, wherein non-hydrophobic accompanying substances are removed using an acidic solution.

19. The method of claim 18, wherein the acidic solution is selected from the group consisting of hydrochloric acid, sulfuric acid, and acetic acid.

20. The method of claim 1, wherein accompanying substances are removed by a process selected from the group consisting of treatment using a solution that removes substances bound through hydrogen bonds, treatment using a solution that removes alkaline sensitive substances, treatment using a solution that removes acid sensitive substances, and any combination thereof.

21. The method of claim 1, wherein the material is of biological origin, and further comprising the step of confectioning the material to a membrane, heart valve, or blood vessel.

22. The method of claim 1, wherein the nativity of the material of biological origin is adjusted to a value below 95%.

23. The method of 22, wherein the nativity of the material of biological origin is adjusted by irradiation with a dose of 5 kGy-50 kGy.

24. The method of claim 1, wherein the material is of biological origin, and further comprising processing the material into a collagen solution, collagen fibers, collagen weaves, collagen fleeces or collagen sponges.

25. The method of claim 1, wherein the material is of biological origin, and further comprising processing the material into collagen-elastin solutions, collagen-elastin fibers, collagen-elastin weaves, collagen-elastin fleeces or collagen-elastin sponges.

26. The method of claim 21, further comprising the step of seeding the confectioned material of biological origin with cells.

27. The method of claim 26, further comprising the step of using the material of biological origin for tissue engineering purposes.

28. A collagen-elastin product in the form of an insoluble collagen which contains insoluble elastin.

29. The collagen-elastin product of claim 28, containing at least 20% (w/w) of insoluble elastin fibers retaining their original fibrillin structure.

30. The collagen-elastin product of claim 28, containing at least 60% (w/w) of insoluble elastin fibers retaining their original fibrillin structure.

31. The collagen-elastin product of claim 28, showing revascularization after implantation.

32. A membrane for tissue engineering produced by the method of claim 1.

33. The membrane of claim 32, used for articular cartilage regeneration of MACI®, ACI, or ACT techniques.

34. The membrane of claim 32 in the form of a collagen-elastin membrane.

35. The membrane of claim 32, containing residues of a marker molecule selected from the group consisting of glucosamine and galactosamine, below 0.5 n/1000 n.

36. The membrane of claim 32, having a nativity which is adjusted to a value below 95%.

37. The membrane of claim 32, promoting cell adhesion and/or proliferation of seeded cells.

38. A product of biological origin obtained from tendons, ligaments, tubular grafts, skins, or membranes for tissue engineering purposes, and produced by the method of claim 1.

39. The product of claim 38, containing residues of a marker molecule selected from the group consisting of glucosamine and galactosamine, below 0.5 n/1000 n.

40. The product of claim 39, processed into a freeze-dried sponge for tissue engineering purposes.
